(19) United States
(12) Patent Application Publication
(10) Pub. No.: US 2020/0397292 A1
(43) Pub. Date: Dec. 24, 2020

(54) DEVICE FOR THE REMOTE MONITORING OF BODY TEMPERATURE IN CATTLE AND PIGS

(71) Applicant: Carlos LEON CABRERA, . (ES)

(72) Inventor: Ignacio Antonio ESPINOSA CRESPO, . (ES)

(21) Appl. No.: 16/969,986

(22) PCT Filed: Apr. 9, 2019
(86) PCT No.: PCT/ES2019/000029

§ 371 Date: Aug. 14, 2020

(30) Foreign Application Priority Data
Feb. 14, 2018 (ES) U201800087

(51) Int. Cl. A61B 5/00; A61B 5/01

(52) U.S. Cl. CPC A61B 5/0008; A61B 5/01; A61B 2560/0214; A61B 2503/40; A61B 2562/16; A61B 5/0022

(57) ABSTRACT

Remote monitoring device for body temperature in bovine and porcine cattle comprised by a fusiform temperature-conductor shell, a circular lid which hermetically closes the device, both coated with a biocompatible material, and an internal unit consisting on a thermic probe assembled to a programmable circuit, which provides data on the unique identification of the animal, which also consists on a radio transmitter, an aerial and the battery feeding the device.

The device can be programmed to take the temperature readings, record and send the information to a receptor, which can transmit the data to any remote device through the 2G network.

### FIELD OF INVENTION

Veterinary and livestock health, more precisely, in the field of diagnostic devices based in body temperature in intensive farming conditions.

### BACKGROUND OF THE INVENTION

The present invention consists on a measuring device which can measure the animal body temperature and transmit the data in remote. Raised temperature in animals is a common clinical sign of a high number of diseases, most of them infectious diseases which may seriously compromise animal health and productivity. Early detection of such temperature raises is key for the improvement of diagnosis, treatment and prognosis of unforeseen disease.

Until now, the temperature tests were carried out sporadically by using clinical thermometers via rectum, without the possibility to keep the animal continuously monitored and requiring continuous handling of the animal.

There are existing external-fixed devices which can take the temperature from the animal auricular area, though these devices tend to get lost due to the natural tendency of the animal to socialising.

There is no existing device that can take the animals temperature and transmit automatically and in real time the temperature data to a receptor located more than 10 meters away, without any human intervention. There are subcutaneous devices which can measure temperature in animals. However, close human intervention is required in order to read temperature measures.

### INVENTION EXPLANATION

The objective of this invention has a non described before purpose. On one hand, describing a device which can be used subcutaneously for both male and/or female, or intravaginal only for females, to minimise the risk of lost. The other objective is to achieve continuous and remote monitoring of the animal body temperature.

The device consists on two different parts, a plastic fusiform shell coated with a biocompatible and temperature conductor coat, variable in size depending on the size of the animal to monitor, by way of guidance, ranging from 8 to 10 centimeters long and a diameter between 1.5 centimeters to 2.5 centimeters in the thickest part.

Inside the shell a thermographic probe is placed and fixed to a programmable circuit, which is also equipped with a memory card, a battery and a transmitter and an aerial, transmitting data via radio to 433 MHz to a receptor located at a maximum distance of 100 meters.

The programmable circuit takes periodical readings of the probe and register the values. When the animal and the receptor are within the reception distance, data recorded from the last reception are sent. The data are sent through a unique identification of the devices which is also recorded in the circuit.

Once the data are received by the receptor, the data are transmitted to any device through the 2G network. The receptor should be placed near the drinking tank points of the animals, so that continuous reception of the data can be ensured, since animals approach for drinking several times a day. This way, remote temperature monitoring is achieved without a daily handling of animals.

Once the device has been introduced inside the animal, either via intravaginal or subcutaneously, the risk of lost is very rare even if the animal scratches or licks or by any other natural behaviour.

### BRIEF DESCRIPTION OF THE DRAWINGS

FIG. 1 represents the plastic fusiform shell

FIG. 2 represents the lid of the plastic shell.

FIG. 3 represents a scheme of the internal unit of the shell.

These figures correspond to the drawings shown in page 5.

### PREFERENT EMBODIMENT OF THE INVENTION

Below there is an example of the device in accordance with the present invention, with references to the figures attached.

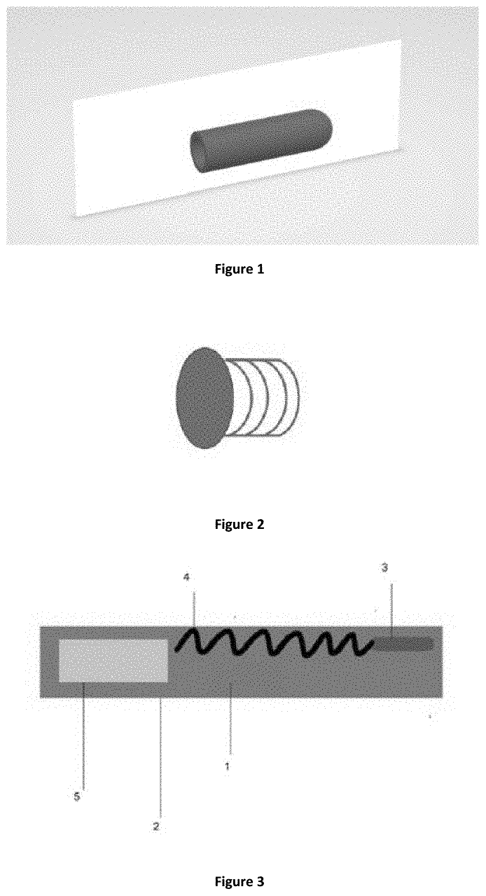

FIG. 1 shows the plastic fusiform shell with roughly 8 centimeters length and a diameter of 2 centimeters with sufficient space to harbour the internal nucleus of the device.

FIG. 2 represents the lid in 2 centimeters of diameter, which hermetically closes the shell.

FIG. 3 is a drawing of the internal nucleus of the device consisting on:
1. A thermic plaque linked by welding to a thermic probe.
2. The thermic probe is assembled to a microcircuit designed and mounted with a transmitter, an aerial and a replaceable battery.

The shell is coated with a biocompatible material coating, through an industrialised process.

1. Device for the remote monitoring of body temperature in bovine and porcine cattle, comprised by a fusiform and temperature transmitting shell (FIG. 1), a circular lid which hermetically closes the device (FIG. 2), both coated with a biocompatible material, and also comprised by an internal unit (FIG. 3) consisting on a thermic probe (1), assembled to a programmable microcircuit (2), which provides the unique identification data of the animal, which is also consisting on a radio transmitter (3), an aerial (4) and a battery (5) feeding the device.

2. As claimed 1 subcutaneous device in male and female.

3. As claimed 1 intravaginal device for female.

4. As claimed 1 device for bovine cattle.

5. As claimed 1 device for porcine cattle.
